(19) United States
(12) Patent Application Publication
(10) Pub. No.: US 2014/0057345 A1
(43) Pub. Date: Feb. 27, 2014

(54) Polypropylene Resin Formulation for Autoclave Applications

(71) Applicants: Mahesh Patkar, Houston, TX (US); Michael McLeod, Kemah, TX (US)

(72) Inventors: Mahesh Patkar, Houston, TX (US); Michael McLeod, Kemah, TX (US)

(73) Assignee: FINA TECHNOLOGY, INC., Houston, TX (US)

(21) Appl. No.: 13/595,390

(22) Filed: Aug. 27, 2012

(51) Int. Cl. C08L 23/16; C12M 1/24; B01L 3/00; B29C 49/08; C08K 5/098

(52) U.S. Cl. 435/304.1; 264/537; 524/399; 422/548

(57) ABSTRACT

A method including supplying a polypropylene resin, blending the polypropylene resin with Zinc Stearate, and forming a molded article from the polypropylene resin. Further, an article including a polypropylene resin blended with Zinc Stearate.

### FIELD

Embodiments of the invention generally relate to polypropylene compositions. In particular, embodiments of the invention relate to polypropylene compositions useful for autoclave applications.

### BACKGROUND

Laboratory and medical grade polymer articles are typically sterilized, often in an autoclave. When these articles are heated in a autoclave, the articles can become hazy or develop microscopic defects, which may appear as white specs.

### SUMMARY

In one embodiment of the present disclosure, a method is disclosed. The method includes supplying a polypropylene resin, blending the polypropylene resin with Zinc Stearate, and forming a molded article from the polypropylene resin.

In another embodiment of the present disclosure, an article is disclosed. The article includes a polypropylene resin blended with Zinc Stearate.

### DETAILED DESCRIPTION

### Introduction and Definitions

It is to be understood that the following disclosure provides many different embodiments, or examples, for implementing different features of various embodiments. Specific examples of components and arrangements are described below to simplify the present disclosure. These are, of course, merely examples and are not intended to be limiting.

Various terms as used herein are shown below. To the extent a term used in a claim is not defined below, it should be given the broadest definition persons in the pertinent art have given that term as reflected in printed publications and issued patents. Further, unless otherwise specified, all compounds described herein may be substituted or unsubstituted and the listing of compounds includes derivatives thereof.

Certain polymerization processes disclosed herein involve contacting polyolefin monomers with one or more catalyst systems to form a polymer.

### Catalyst Systems

Catalyst systems useful for polymerizing olefin monomers include any catalyst system capable of forming a polymer composition exhibiting the claimed properties. For example, the catalyst system may include single site transition metal catalyst systems including metallocene catalyst systems, Ziegler-Natta catalyst systems or combinations thereof, for example. The catalysts may be activated for subsequent polymerization and may or may not be associated with a support material, for example. A brief discussion of such catalyst systems is included below, but is in no way intended to limit the scope of the invention to such catalysts.

In one or more specific embodiments, the polymer composition is formed by a metallocene catalyst system. Metallocene catalysts may be characterized generally as coordination compounds incorporating one or more cyclopentadienyl (Cp) groups (which may be substituted or unsubstituted, each substitution being the same or different) coordinated with a transition metal through π bonding. The substituent groups on Cp may be linear, branched or cyclic hydrocarbyl radicals, for example. The cyclic hydrocarbyl radicals may further form other contiguous ring structures, including indenyl, azulenyl and fluorenyl groups, for example. These contiguous ring structures may also be substituted or unsubstituted by hydrocarbyl radicals, such as C₁ to C₂₀ hydrocarbyl radicals, for example.

In other embodiments Ziegler Natta catalyst systems may be used. Ziegler-Natta catalyst systems are generally formed from the combination of a metal component (e.g., a catalyst) with one or more additional components, such as a catalyst support, a cocatalyst and/or one or more electron donors, for example.

### Polymerization Processes

As indicated elsewhere herein, catalyst systems are used to make polyolefin compositions. Once the catalyst system is prepared, as described above and/or as known to one skilled in the art, a variety of processes can be carried out using that composition. Among the varying approaches that can be used include procedures set forth in U.S. Pat. No. 5,525,678, incorporated by reference herein. The equipment, process conditions, reactants, additives and other materials will of course vary in a given process, depending on the desired composition and properties of the polymer being formed. For example, the processes of U.S. Pat. No. 6,420,580, U.S. Pat. No. 6,380,328, U.S. Pat. No. 6,359,072, U.S. Pat. No. 6,346,586, U.S. Pat. No. 6,340,730, U.S. Pat. No. 6,339,134, U.S. Pat. No. 6,300,436, U.S. Pat. No. 6,274,684, U.S. Pat. No. 6,271,323, U.S. Pat. No. 6,248,845, U.S. Pat. No. 6,245,868, U.S. Pat. No. 6,245,705, U.S. Pat. No. 6,242,545, U.S. Pat. No. 6,211,105, U.S. Pat. No. 6,207,606, U.S. Pat. No. 6,180,735 and U.S. Pat. No. 6,147,173 may be used and are incorporated by reference herein.

The catalyst systems described above can be used in a variety of polymerization processes, over a wide range of temperatures and pressures. The temperatures may be in the range of from about −60° C. to about 280° C., or from about 50° C. to about 200° C. and the pressures employed may be in the range of from 1 atmosphere to about 500 atmospheres or higher.

Polymerization processes may include solution, gas phase, slurry phase, high pressure processes or a combination thereof.

In certain embodiments, the process of the invention is directed toward a solution, high pressure, slurry or gas phase polymerization process of one or more olefin monomers having from 2 to 30 carbon atoms, or from 2 to 12 carbon atoms or from 2 to 8 carbon atoms, such as ethylene, propylene, butane, pentene, methylpentene, hexane, octane and decane. Other monomers include ethylenically unsaturated monomers, diolefins having from 4 to 18 carbon atoms, conjugated or nonconjugated dienes, polyenes, vinyl monomers and cyclic olefins. Non-limiting monomers may include norbornene, nobornadiene, isobutylene, isoprene, vinylbenzocyclobutane, sytrenes, alkyl substituted styrene, ethylidene norbornene, dicyclopentadiene, and cyclopentene. In one embodiment, a copolymer is produced, such as propylene/ethylene, or a terpolymer is produced. Examples of solution processes are described in U.S. Pat. No. 4,271,060, U.S. Pat. No. 5,001,205, U.S. Pat. No. 5,236,998 and U.S. Pat. No. 5,589,555, which are incorporated by reference herein.

One example of a gas phase polymerization process generally employs a continuous cycle, wherein a cycling gas stream (otherwise known as a recycle stream or fluidizing medium) is heated in a reactor by heat of polymerization. The heat is removed from the recycle stream in another part of the cycle by a cooling system external to the reactor. The gaseous stream containing one or more monomers may be continuously cycled through a fluidized bed in the presence of a catalyst under reactive conditions. The gaseous stream is withdrawn from the fluidized bed and recycled back into the reactor. Simultaneously, polymer product is withdrawn from the reactor and fresh monomer is added to replace the polymerized monomer. (See, for example, U.S. Pat. No. 4,543,399, U.S. Pat. No. 4,588,790, U.S. Pat. No. 5,028,670, U.S. Pat. No. 5,317,036, U.S. Pat. No. 5,352,749, U.S. Pat. No. 5,405,922, U.S. Pat. No. 5,436,304, U.S. Pat. No. 5,456,471, U.S. Pat. No. 5,462,999, U.S. Pat. No. 5,616,661 and U.S. Pat. No. 5,668,228, which are incorporated by reference herein.)

The reactor pressure in a gas phase process may vary from about 100 psig to about 500 psig, or from about 200 psig to about 400 psig or from about 250 psig to about 350 psig, for example. The reactor temperature in a gas phase process may vary from about 30° C. to about 120° C., or from about 60° C. to about 115° C., or from about 70° C. to about 110° C. or from about 70° C. to about 95° C. Other gas phase processes contemplated by the process includes those described in U.S. Pat. No. 5,627,242, U.S. Pat. No. 5,665,818 and U.S. Pat. No. 5,677,375, which are incorporated by reference herein.

Slurry processes generally include forming a suspension of solid, particulate polymer in a liquid polymerization medium, to which monomers and optionally hydrogen, along with catalyst, are added. The suspension (which may include diluents) can be intermittently or continuously removed from the reactor where the volatile components can be separated from the polymer and recycled, optionally after a distillation, to the reactor. The liquefied diluent employed in the polymerization medium is typically an alkane having from 3 to 7 carbon atoms, such as a branched alkane. The medium employed is generally liquid under the conditions of polymerization and relatively inert. Such as hexane or isobutene.

A slurry process or a bulk process (e.g., a process without a diluent) may be carried out continuously in one or more loop reactors. The catalyst, as a slurry or as a dry free flowing powder, can be injected regularly to the reactor loop, which can itself be filled with circulating slurry of growing polymer particles in a diluent. Hydrogen, optionally, may be added as a molecular weight control. The reactor may be maintained at a pressure of from about 27 bar to about 45 bar and a temperature of from about 38° C. to about 121° C., for example. Reaction heat can be removed through the loop wall since much of the reactor is in the form of a double-jacketed pipe. The slurry may exit the reactor at regular intervals or continuously to a heated low pressure flash vessel, rotary dryer and a nitrogen purge column in sequence form removal of the diluent and all unreacted monomer and comonomers. The resulting hydrocarbon free powder can then be compounded for use in various applications. Alternatively, other types of slurry polymerization processes can be used, such stirred reactors is series, parallel or combinations thereof. Upon removal from the reactor, the polymer may be passed to a polymer recovery system for further processing, such as addition of additives and/or extrusion, for example.

### Polymer Product

The polymers produced by the processes described herein may include propylene based polymers (e.g., polypropylene and polypropylene copolymers), for example. The polypropylene copolymers may include propylene-ethylene copolymers.

The polymers may have a narrow molecular weight distribution (M_{w}/Mₙ). As used herein, the term “narrow molecular weight distribution” refers to a polymer having a molecular weight distribution of from about 1.5 to about 8, or from about 2.0 to about 7.5 or from about 2.0 to about 7.0, for example.

In one embodiment, propylene based polymers may have a recrystallization temperature (Tᵣ) of less than 130° C., or from 110° C. to 125° C., or 120° C., for example.

The propylene based polymers may have a melting point, also referred to as second melt peak, (Tₘ) (as measured by DSC) of at least 120° C., or from 120° C. to 175° C., or from 135° C. to 165° C., or from 140° C. to 150° C., for example.

The propylene based polymers may include about 15 wt. % or less, or about 12 wt. % or less, or about 10 wt. % or less, or about 6 wt. % or less, or about 5 wt. % or less or about 4 wt. % or less, or from 0.1 wt. % to 2 wt. %, or from 0.2 wt. % to about 1 wt. % of xylene soluble material (XS), for example (as measured by ASTM D5492-06).

The propylene based polymers may have a melt flow rate (MFR) (as measured by ASTM D-1238) of from 0.01 dg/min to 1000 dg/min., or from 0.01 dg/min. to 100 dg/min., or from 10 dg/min. to 60 dg/min., or from 20 dg/min to 50 dg/min., or from 30 dg/min. to 40 dg/min., for example.

In one or more embodiments, the polymers include propylene based random copolymers. Unless otherwise specified, the term “propylene based random copolymer” refers to those copolymers composed primarily of propylene and an amount of at least one comonomer, wherein the polymer includes at least about 0.2 wt. %, or at least about 0.8 wt. %, or at least about 2 wt. %, or from about 0.1 wt. % to about 5.0 wt. %, or from about 0.4 wt. % to about 1.0 wt. % comonomer relative to the total weight of polymer, for example.

The comonomers may be selected from C₂ to C₁₀ alkenes. For example, the comonomers may be selected from ethylene, propylene, 1-butene, 1-pentene, 1-hexene, 1-heptene, 1-octene, 1-nonene, 1-decene, 4-methyl-1-pentene and combinations thereof. In one specific embodiment, the comonomer includes ethylene. Further, the term “random copolymer” refers to a copolymer formed of macromolecules in which the probability of finding a given monomeric unit at any given site in the chain is independent of the nature of the adjacent units.

The propylene based random copolymers may exhibit a melt flow rate of at least 2 dg./10 min., or from 5 dg./10 min. to 60 dg./10 min. or from 10 dg./10 min. to 50 dg./10 min., or from 20 dg/min. to 45 dg/min., or from 30 dg/min. to 40 dg/min., for example.

### Product Application

The polymers produced are useful in a variety of end-use applications, such as molded articles. More particularly, the polymers can be used for laboratory and medical end-use articles, such as pipette tips and centrifuge tubes.

In one embodiment, the polymer is used to form a molded article, such as, a medical or laboratory grade article. For example, the molded article may include, but is not limited to, a pipette tip, centrifuge tube, reaction vessel, protein assay trays, syringe, petri dish or culture tube. The molded article may be produced using any method known to those of ordinary skill in the art, such as blow molding, compression molding, injection stretch blow molding, etc.

In order to modify or enhance certain properties of the molded articles for specific end-uses, certain molded articles include an acid neutralizer to neutralize corrosive acidic residues remaining in the polymer. Neutralizing these acidic residues may result in melt flow improvement and improved melt flow stability. Traditional acid neutralizers include calcium stearate, hydrotalcite, calcium oxide and Pationic 940 (a modified calcium salt derived from stearic and lactic acids). Use of these traditional acid neutralizers for molded articles that are autoclaved may result in increased haze and microscopic defects, which may appear as white spots in the molded article after autoclaving.

In certain embodiments of the present disclosure, zinc stearate (ZnSt) is used as an acid neutralizer. Unexpectedly, use of ZnSt as an acid neutralizer in place of a traditional acid neutralizer, reduces or eliminates microscopic defects in the molded articles and results in improved haze after autoclaving as compared to traditional acid neutralizers. In certain embodiments, ZnSt may be added in an amount from 0.01 to 0.15 wt %, or from 0.05 to 0.10 wt %, or about 0.07 wt %. In certain embodiments of the present disclosure, the only acid neutralizer compound added to the polymer fluff is ZnSt. In certain other embodiments of the present disclosure, modified calcium salts such as modified calcium salts derived from stearic and lactic acids, for example, Pationic 940 (commercially available from PATCO Polymer Additives), is not added to the polymer fluff. In another embodiment, an amine oligomer, such as a sterically hindered amine oligomer, for example, Uvinul 5050H (commercially available from BASF).

Haze values of articles after autoclaving in certain embodiments of the present disclosure are dependent upon haze values of the articles before autoclaving and the thickness of the article itself. In certain embodiments of the present disclosure, the haze of a polypropylene article with a thickness of 0.5 mm made from polymer fluff having ZnSt as the acid neutralizer was less than about 20 or between about 10 and about 20. In other embodiments, the haze of a polypropylene article with a thickness of 1 mm made from polymer fluff having ZnSt as the acid neutralizer was less than about 35 or between about 25 and about 33. In other embodiments, the haze of a polypropylene article with a thickness of 15 mm made from polymer fluff having ZnSt as the acid neutralizer was less than about 45 or between about 40 and about 45. Haze values are determined by ASTM D1003 procedure “A.”

Other additives may include stabilizers (e.g., hindered amines, benzofuranon, indolinone) to protect against UV degradation, thermal or oxidative degradation and/or actinic degradation, anti-blocks, coefficient of friction modifiers, processing aids, colorants, clarifiers, nucleators, radiation additives, and other additives known to those skilled in the art. In one embodiment, the additives which are useful are those that are not surface active additives or that do not migrate to the surface of the polymer or article. Examples of such additives, such as clarifiers and nucleators, include, but are not limited to, Millad® 3988, Millad® NX8000 (commercially available from Milliken Chemicals), ADK NA-21 and ADK NA-71 (commercially available from Amfine Chemicals), and CGX386 (commercially available from Ciba). More generally, clarifiers may include inorganic nucleating agents (pulverized clay, silicates, alkali salts, alkaline earth salts, aluminum salts, titanium salts, and metal oxides, for example), organic nucleating agents (2-mercaptobenzimidazole, sorbitol derivatives, and phosphate derivates, for example), and 1,3,5-trisamide derivatives. Clarifiers may be added in the range of from 0.10 wt % to 0.4 wt %, or from 0.15 wt % to 0.25 wt %, for example. Processing aids, such as Irgafos 168 (available from Ciba) and others known to those skilled in the art, may be added in the range of from 0.05 wt % to 0.20 wt. %, or from 0.1 wt. % to 0.15 wt. %, for example.

In one embodiment, the additive is a radiation additive which provides radiation resistance during the sterilization process of the molded article. Examples of such additives include Chimmasorb 944 and Tinuvin 622 (commercially available from Ciba), and other such similar additives, such as hindered amines and oligomeric hindered amines. The additives may be combined with the polymer during the processing phase (pellet extrusion), for example. It is further contemplated that multiple radiation additives may be used in combination. The total amount of radiation additive may be added in an amount less than 0.25 wt %, or from 0.05 wt % to 0.20 wt. %, or from 0.1 wt. % to 0.15 wt. %, based on the total weight of the polymer.

The molded article may exhibit a flexural modulus of from 100 to 350 kpsi, or from 150 to 300 kpsi, or from 200 to 250 kpsi. or from 160 to 200 kpsi, for example.

### EXAMPLES

Polypropylene resin fluff made using a Ziegler-Natta catalyst and including 0.6 wt % ethylene as a comonomer was mechanically blended with additives as shown in Table 1. Table values in Table 1 are given in weight percent. Sample No. N11044-1 was Total 3847MR. The resulting resin was formulated into articles and autoclaved at 131° C. for 30 minutes. Results are shown in Table 2. A ByK Gardner Hazegard Plus System was used to determine haze using ASTM D1003 procedure “A”. The examination for defects was performed microscopically.

TABLE 1

| | Sample No. | | | |
|---|---|---|---|---|
| Additive | N11044-1 | N11044-2 | N11044-3 | N11044-4 |
| Univul 5050H | .09 | | 0.09 | |
| Doverphos | .03 | .03 | .03 | .03 |
| S-9228T | | | | |
| Pationic 940 | .03 | .03 | | |
| CIBA XT386 | .02 | .02 | .02 | .02 |
| Peroxide | As Needed | As Needed | As Needed | As Needed |
| ZnSt | | | .07 | .07 |
| Tinuvin 622 | | .08 | | .08 |

TABLE 2

| Sample No. | N11044-1 | N11044-2 | N11044-3 | N11044-4 |
|---|---|---|---|---|
| Defects after Autoclaving | Yes | Yes | No | No |
| Haze (.02 inch thickness) | 5.52/21.3 | 5.90/20.3 | 5.53/10.8 | 5.75/18.4 |
| (before/after) autoclaving | | | | |
| Haze (.04 inch thickness) | 15.3/33.5 | 16.1/37.0 | 16.2/25.3 | 16/32.6 |
| (before/after) autoclaving | | | | |
| Haze (.06 inch thickness) | 28.9/46.1 | 30.6/53.2 | 31.0/42.1 | 29.1/44.4 |
| (before/after) autoclaving | | | | |

The replacement of Pationic 940 with ZnSt results in a significant improvement in both reducing defects and limiting the increase in haze during autoclaving. Further, in each case, the use of ZnSt resulted in a decreased haze after autoclaving in comparison to Pationic 940.

While embodiments have been shown and described, modifications thereof can be made by one skilled in the art without departing from the spirit and teachings of the disclosure. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from 1 to 10 includes, 2, 3, 4, etc.; greater than 0.10 includes 0.11, 0.12, 0.13, etc.). For example, whenever a numerical range with a lower limit, R_{L}, and an upper limit, R_{U}, is disclosed, any number falling within the range is specifically disclosed. In particular, the following numbers within the range are specifically disclosed: R=R_{L}+k*(R_{U}-R_{L}), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference is not an admission that it is prior art to the present invention, especially any reference that may have a publication date after the priority date of this application. The disclosures of all patents, patent applications, and publications cited herein are hereby incorporated by reference, to the extent that they provide exemplary, procedural, or other details supplementary to those set forth herein.

1. A method comprising:
supplying a polypropylene resin comprising 6 wt. % or less xylene soluble material as measured by ASTM D5492-06;
blending the polypropylene resin with Zinc Stearate;
forming a molded article from the polypropylene resin; and
autoclaving the molded article, wherein, after autoclaving, the molded article exhibits a haze of between about 10 and about 20 as determined by ASTM D1003 procedure “A”.

2. The method of claim 1, wherein the polypropylene resin comprises from 0.1 wt. % to 2 wt. % xylene soluble material.

3. The method of claim 1, wherein the autoclaving is performed at a temperature of about 121° C.

4. The method of claim 1, wherein the step of forming the molded article is performed by blow molding, compression molding, or injection stretch blow molding.

5. The method of claim 1 further comprising before the step of supplying the polypropylene resin:
forming a polypropylene resin by polymerizing a propylene monomer using a Ziegler-Natta catalyst.

6. The method of claim 1, wherein the polypropylene is a copolymer.

7. The method of claim 6, wherein the copolymer is a propylene/ethylene copolymer.

8. An article comprising:
a polypropylene resin comprising 6 wt. % or less xylene soluble material as measured by ASTM D5492-06, wherein the polypropylene resin is blended with Zinc Stearate, and wherein, after autoclaving, the article exhibits a haze of between about 10 and about 20 as determined by ASTM D1003 procedure “A”.

9. The article of claim 8, wherein the article is a molded article.

10. The article of claim 9, wherein the molded article is a pipette tip, centrifuge tube, reaction vessel, protein assay trays, syringe, petri dish or culture tube.

11. The article of claim 8, wherein the Zinc Stearate is present in the resin in a range of from 0.01 to 0.15 wt. %.

12. The article of claim 9, wherein the Zinc Stearate is present in the resin an amount of about 0.07 wt. %.

13. The article of claim 8, wherein the article has a radiation additive.

14. The article of claim 8, wherein the article does not include any modified calcium salts.

15. The article of claim 8, wherein Zinc Stearate is the only acid neutralizer in the polypropylene resin.

16. The article of claim 8, wherein the polypropylene resin comprises from 0.1 wt. % to 2 wt. % xylene soluble material.

17. The article of claim 8 further includes an amine oligomer.

18. The article of claim 8, wherein the polypropylene resin is a copolymer.

19. The article of claim 18, wherein the polypropylene resin is 0.06% by weight ethylene.

20. The article of claim 8, wherein no specs are visible from a visual inspection.

21. The article of claim 18, wherein the polypropylene resin is 0.6% by weight ethylene.
